(19) United States
(12) Patent Application Publication
(10) Pub. No.: US 2010/0267988 A1
(43) Pub. Date: Oct. 21, 2010

(54) PROCESSES FOR PREPARING INTERMEDIATE COMPOUNDS USEFUL FOR THE PREPARATION OF CINACALCET

(75) Inventors: Tibor Szekeres, Budapest (HU); Jozsef Repasi, Budapest (HU); Andras Szabo, Budapest (HU); Bernardino Mangion, Santa Lucia (MT)

Correspondence Address: IP Patent Docketing;K&L GATES LLP, 599 Lexington Avenue, 33rd Floor, New York, NY, 10022-6030, US

(73) Assignee: Medichem, S.A., Barcelona (ES)

(21) Appl. No.: 12/303,903

(22) PCT Filed: Jun. 8, 2007
(86) PCT No.: PCT/IB07/03346

§ 371 Date: May 19, 2010

Related U.S. Application Data
(60) Provisional application No. 60/811,786, filed on Jun. 8, 2006.

(51) Int. Cl. C07C 211/30; C07C 45/38; C07C 29/44

(52) U.S. Cl. 564/387; 568/431; 568/812

(57) ABSTRACT

The invention relates, in general, to an improved process for preparing compounds (e.g., 3-(3-trifluoromethylphenyl)propanal (Compound III, below)), which are key intermediates for the synthesis of cinacalcet, its salts and/or solvates thereof, as well as the use of such compounds prepared by such process for the preparation of cinacalcet and/or its salts or solvates.

### CROSS REFERENCE TO RELATED APPLICATIONS

This application claims priority to U.S. Provisional Application No. 60/811,786, filed Jun. 8, 2006, application which is expressly incorporated herein by reference in its entirety.

### BACKGROUND OF THE INVENTION

1. Field of the Invention

The invention relates, in general, to an improved process for preparing compounds (e.g., 3-(3-trifluoromethylphenyl)propanal (Compound III, below)), which are key intermediates for the synthesis of cinacalcet, its salts and/or solvates thereof, as well as the use of such compounds prepared by such process for the preparation of cinacalcet and/or its salts or solvates.

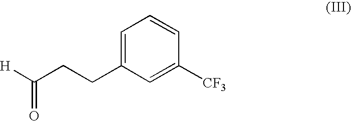

2. Discussion of the Related Art

Cinacalcet is a commercially marketed pharmaceutically active substance known to be useful for the treatment of secondary hyperparathyroidism in patients with chronic kidney disease on dialysis and for the treatment of hypercalcemia in patients with parathyroid carcinoma. Cinacalcet is the international commonly accepted name for N-[1-(R)-(−)-1-naphthyl)ethyl]-3-[3-(trifluoromethyl)phenyl]-1-aminopropane hydrochloride, which has an empirical formula of C₂₂H₂₂F₃N.HCl, a molecular weight of 393.9 and has the structural formula (I):

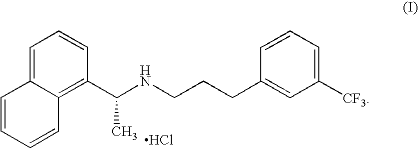

U.S. Pat. No. 6,011,068 generally describes cinacalcet and its pharmaceutically acceptable acid addition salts, but does not provide any examples for the preparation of the same.

U.S. Pat. No. 6,211,244 describes cinacalcet and its pharmaceutically acceptable acid chloride addition salt, but does not provide any examples for the preparation of cinacalcet and/or cinacalcet hydrochloride.

Drugs 2002, 27(9), 831-836 discloses a synthetic scheme for preparing cinacalcet hydrochloride according to the general procedure described in U.S. Pat. No. 6,211,244. This disclosed synthetic route is illustrated in Scheme 1, below.

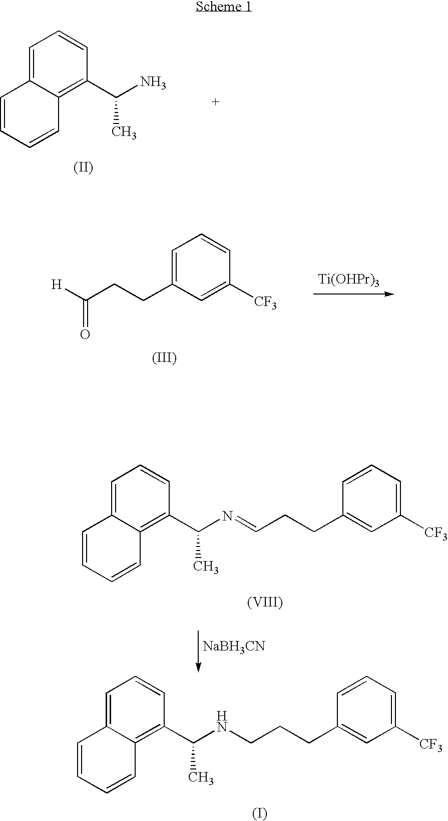

In this regard, several processes for preparing Compound III (i.e., 3-(3-trifluoro methylphenyl)propanal) have been described in the literature.

European Patent EP 0 194 764 discloses a process for preparing Compound III in which Compound IV (i.e., 3-trifluoromethylbromobenzene) is reacted with Compound V (i.e., propargyl alcohol) using bis(triphenylphosphine)palladium chloride and cuprous iodide in triethylamine, followed by catalytic hydrogenation to give the corresponding alcohol (compound VII). Compound VII is then converted to Compound III by a Swern oxidation. This synthetic procedure is illustrated in Scheme 2, below.

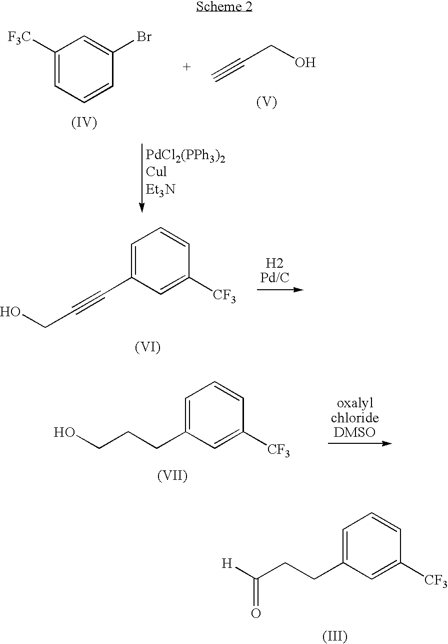

In Tetrahedron Letters 2004, 45(45), 8355-58, Compound III is prepared from Compound IX (i.e., 3-trifluoromethylcinnamic acid) by reduction of the double bond and reduction of the carboxylic acid group into the corresponding alcohol followed by a Swern oxidation reaction, as illustrated in Scheme 3, below.

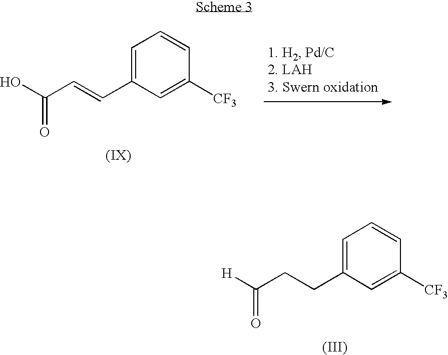

An alternative process for preparing Compound III is described in Journal of Medicinal Chemistry 1968, 11, 1258-62.

### SUMMARY OF THE INVENTION

The invention relates, in general, to an improved process for preparing compounds (e.g., 3-(3-trifluoromethylphenyl)propanal (Compound III)), which are key intermediates for the synthesis of cinacalcet, its salts and/or solvates thereof, as well as the use of such compounds prepared by such process for the preparation of cinacalcet and/or its salts or solvates.

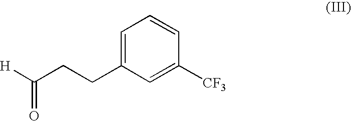

The invention provides an improved process for preparing Compound III. Namely, the process of the invention for preparing Compound III and similar compounds obviates the need to employ a Swern oxidation step (as required in the above-described processes) and therefore avoids the need to employ low temperature oxidation reactions as well as the unpleasant odors associated with such procedures. In particular, the process of the invention includes oxidation of Compound VII with an oxidizing agent using a nitroxyl compound as catalyst in an inert solvent.

The invention further includes a process for preparing Compound VII from compound VI. The invention further provides a process for preparing Compound VI (i.e., 3-(3-trifluoromethylphenyl)propynol) using lower amounts of catalyst and in which the catalyst can be at least partially recycled.

The processes of the invention are clean, fast, have high volume efficacy and require no chromatographic purifications. These characteristics of the processes of the invention make them very suitable for industrial scale up.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The invention relates, in general, to an improved process for preparing compounds (e.g., 3-(3-trifluoromethylphenyl)propanal (Compound III)), which are key intermediates for the synthesis of cinacalcet, its salts and/or solvates thereof, as well as the use of the such compounds prepared such process for the preparation of cinacalcet and/or its salts or solvates.

In particular, the process of the invention includes oxidation of Compound VII with an oxidizing agent using a nitroxyl compound as catalyst in an inert solvent to yield Compound III. A suitable nitroxyl compound for use in the invention includes TEMPO (2,2,6,6,-tetramethy-1-piperidinyloxy free radical). A suitable oxidation agent for use in the invention includes sodium hypochlorite. Suitable inert solvents for use in the invention include any solvent that does not take part in the reaction. Preferred inert solvents include, for example, cyclic or acyclic alkanes (e.g., hexane, heptane, methylcyclohexane), aromatic solvents (e.g., toluene), halogenated solvents (e.g., dichloromethane, dichloroethane, chloroform), esters (e.g., ethyl acetate, butyl acetate, isopropyl acetate) or ethers (e.g., diethyl ether, tetrahydrofuran or tert-butyl methyl ether) and/or mixtures thereof.

Preferably, the oxidation reaction is performed using between approximately 0.9 to approximately 2.0 moles of sodium hypochlorite per mol of Compound VII, preferably approximately 1.05 moles. It was furthermore found to be advantageous to add the sodium hypochlorite in portions to the reaction mixture. Preferably, approximately 1 mole of sodium hypochlorite per mol of Compound VII was added to the reaction mixture in a first portion, and after a period of stirring, a second portion of approximately 0.05 moles of sodium hypochlorite per mol of Compound VII was added.

The reaction can optionally be performed using potassium bromide as a regenerating agent of the nitroxyl compound used as catalyst.

Preferably, the oxidation reaction is conducted using a range of temperatures of approximately 5° C. to approximately 25° C. and for a time of approximately 10 to approximately 60 minutes. More preferably below 15° C., and for a time of approximately 20 to approximately 60 minutes.

Optionally, Compound III can be treated with sodium bisulphite to obtain a bisulphite adduct that can be further converted to a purified Compound III. Alternatively, Compound III can be purified by distillation under vacuum.

Compound VII can be obtained according to the process described in the European Patent EP 0 194 764 (see Scheme 2, above). Alternatively, the reaction of Compound IV with Compound V (i.e., propargyl alcohol) can be performed using 10% Pd/C catalyst, biphenyl phosphine, copper (I) iodide and diisopropylamine, to yield Compound VI. Compound VI can readily be converted to Compound VII via catalytic hydrogenation in the presence of Pd/C catalyst.

Another aspect of the invention includes the use of Compound III obtained according to the above-described processes for producing cinacalcet and/or its pharmaceutically acceptable salts and/or solvates thereof.

The various embodiments of the invention having thus been generally described, several examples will hereafter be discussed to illustrate the inventive aspects more fully.

### SPECIFIC EXAMPLES

The following examples are for illustrative purposes only and are not intended, nor should they be interpreted to, limit the scope of the invention.

General Experimental Conditions: Gas Chromatography Method

The gas chromatographic separation was carried out using a RTX-50, 30 m×0.32 mm×0.25 μm column, a head pressure of 10 psi and helium as the carrier gas. Temperature program: 60° C. (2 minute)-10° C./minute-100° C. (0 minute)-20° C./minute-250° C. (10 minutes), Injector temperature: 200° C. Detector (FID) temperature: 250° C.

### Example 1

### Preparation of 3-(3-trifluoromethylphenyl)propanal

Step 1: Preparation of Compound VI (i.e., 3-(3-trifluoromethyl phenyl)propynol)

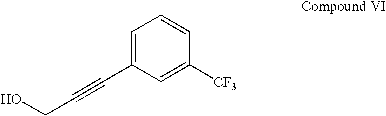

Under argon atmosphere, 266.4 g (1184 mmol) of 3-trifluoromethylbromobenzene, 85.0 g (1516 mmol) of propargyl alcohol, 118.4 g (1.4 mol) of diisopropyl amine, 22.53 g (0.118 mol) of copper (I) iodide, 4.73 g (4.44 mmol) of 10% Pd/C (Selcat Q6) and 31.5 g (0.118 mol) of triphenyl phosphine were dispersed in 1000 mL of distilled water. The reaction mixture was then stirred and refluxed overnight, and the conversion was checked by GC. Next, the reaction mixture was cooled to room temperature (20-25° C.), and 400 mL of tert-butyl methyl ether was added. The resulting mixture was then filtered through a celite pad, and the filtrate was separated. The aqueous layer was then washed two times with 200 mL of tert-butyl methyl ether, and the collected organic layers were dried and evaporated to yield 260 g of crude Compound VI as a dark oil. The resulting crude Compound VI was then purified by vacuum distillation. Purity by GC was approximately 83%. NMR data (200 MHz, CDCl₃, ppm): 3.09 (s, 1H), 4.52 (s, 21-1), 7.39-7.46 (m, 1H), 7.52-7.59 (m, 2H), 7.67 (s, 1H).

Step 2: Preparation of Compound VII (i.e., 3-(3-trifluoromethylphenyl)propan-1-ol

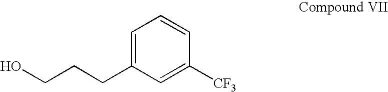

To a solution of 57.5 g (288 mmol) of Compound VI in 60 mL of methanol, 1.4 g of 10% Pd/C (Selcat Q6) was added. The reaction mixture was hydrogenated at a temperature of 42-45° C. and a pressure of 5 bars until all starting material was reacted (approximately 5 hours). The catalyst was removed by filtration and washed with a small amount of methanol. The solution obtained was then evaporated in vacuo. The resulting crude product (49.4 g, 84.1%) was purified by vacuum distillation to yield 41.9 g (Yield: 71.2%) of pure Compound VII product as a nearly colorless oil (bp.: 58-60° C./1.1-1.5 mbar). ¹H NMR data (200 MHz, CDCl₃, ppm): 1.87 (q, 2H), 2.73 (t, 2H), 3.37 (s, 1H), 3.64 (t, 2H), 7.33-7.45 (m, 4H).

Step 3: Preparation of Compound III (i.e., 3-(3-trifluoromethylphenyl)propanal)

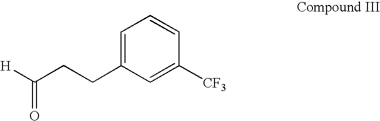

To a solution of 10 g (48 mmol) of Compound VII, 76.6 mg of TEMPO (2,2,6,6-tetramethyl-1-piperidinyloxy free radical) and 234 mg of potassium bromide in 70 mL methylene chloride was added 220 mL (pH=9.5) of sodium hypochlorite solution over 20 minutes at 10-15° C. with stirring. After five minutes of additional stirring, the organic layer was separated. The aqueous layer was extracted twice with 40 mL of methylene chloride, and the collected organic layers were dried and evaporated to yield 10 g of crude Compound III as a yellowish liquid. Yield: 100%; purity (determined by GC): 90.3%, contains 8.25% of Compound NMR data (200 MHz, CDCl₃, ppm): 2.81 (t, 2H), 3.00 (t, 21-1), 7.37-7.46 (m, 4H), 9.80 (s, 1H).

### Example 2

### Preparation of 3-(3-trifluoromethylphenyl)propanal

Step 1: Preparation of Compound VI (i.e., 3-(3-trifluoromethylphenyl)propynol)

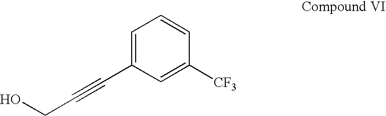

Under argon atmosphere, 23.0 g (102.2 mmol) of 3-trifluoromethylbromobenzene, 7.34 g (130.85 mmol) of propargyl alcohol, 15.32 g (182.83 mmol) of diisopropyl amine, 2.92 g (15.3 mmol) of copper (I) iodide, 0.61 g (0.570 mmol) of 10% Pd/C (Selcat Q6) and 4.02 g (15.32 mmol) of biphenyl phosphine were dispersed in 80 mL of distilled water. The reaction mixture was then stirred and refluxed overnight, and the conversion was checked by GC. Next, the reaction mixture was cooled to room temperature (20-25° C.), and 40 mL of tert-butyl methyl ether were added. The resulting mixture was then filtered through a celite pad, and the filtrate was separated. The aqueous layer was then washed two times with 50 mL of tert-butyl methyl ether, and the collected organic layers were dried and evaporated to yield 45.3 g of crude Compound VI as a dark oil. The resulting crude Compound VI was then purified by vacuum distillation. In this way 14.7 g of the product were obtained. b.p. 120-125° C./3.2-3.8 mbar.

Step 2: Preparation of Compound VII (i.e., 3-(3-trifluoromethylphenyl)propan-1-ol

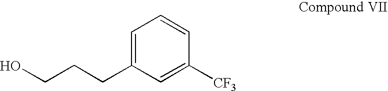

To a solution of 14.5 g (72.5 mmol) of purified 3-(3-trifluoromethylphenyl) propynol in 50 mL of 2-propanol, 0.38 g of 10% Pd/C (Selcat Q6) was added. The reaction mixture was hydrogenated at a temperature of 42-45° C. and a pressure of 5 bars until all starting material was reacted (approximately 5 hours). The catalyst was removed by filtration and washed with a small amount of 2-propanol. The obtained solution was then evaporated in vacuo. The resulting crude product (14.5 g, 100.0%) was purified by vacuum distillation to yield 10.5 g (Yield: 72.1%) of pure Compound VII product as a nearly colorless oil (bp.: 58-60° C./1.1-1.5 mbar).

Step 3:Preparation of Compound III (i.e., 3-(3-trifluoromethylphenyl)propanal

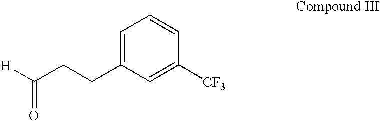

To a mixture of the solution of 10 g (48 mmol) of Compound VII, 76.6 mg of TEMPO (2,2,6,6-tetramethyl-1-piperidinyloxy free radical) in 70 mL of toluene and the solution of 234 mg of potassium bromide in 8 mL of water, 93 mL (pH=9.5) of sodium hypochlorite solution were added over 10 minutes at 10-15° C. with stirring. After five minutes of additional stirring, the organic layer was separated. The aqueous layer was extracted twice with 30 mL of toluene, and the collected organic layers were stirred with a solution of 71 g of sodium bisulphite in 100 mL of water at 100-110° C. until all of the aldehyde bisulphite adduct was separated as white solid. The precipitated adduct was filtered, was suspended two times with 20 mL of toluene and was dried in vacuum to yield 14.8 g of the aldehyde adduct, which was used up without further purification.

Next, 8.86 g (30.4 mmol) of the bisulphite adduct was suspended in 20 mL of water, and 40 mL of 10% sodium hydroxide solution were added with stirring until all solids were dissolved. The obtained opaque solution was extracted six times with 20 mL of dichloromethane. The collected organic layers were dried and evaporated. In this way 4.46 g (71.9%) of the free aldehyde were obtained. Purity: 99.5%.

Alternatively, when the above reaction mixture was not treated with sodium bisulphite then the aldehyde solution was evaporated and the obtained 8.5 g (87.6%) of the crude aldehyde were distilled in fine vacuum. In this way 5.14 g (53.0%) of the aldehyde were obtained as slightly yellowish oil. Purity: 98.0%, b.p. 53-54° C./2.3-2.5 mbar.

### Example 3

### Large scale preparation of Compound III (i.e., 3-(3-trifluoromethyl phenyl)propanal

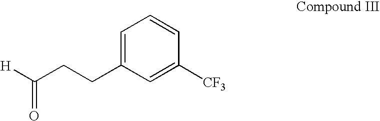

In a 630 L glass-lined reaction vessel, purged with nitrogen and equipped with a pitched blade impeller, were added (in sequence): 0.74 Kg (6.2 mol) of potassium bromide, 0.24 Kg (1.5 mol) of TEMPO (2,2,6,6-tetramethyl-1-piperidinyloxy free radical), 137 Kg of toluene, 31.6 Kg (155 mol) of Compound VII, 68 Kg of toluene, and 29 Kg of water. The mixture was stirred and cooled to 0-5° C. to give a brownish solution. To this mixture, 115 Kg (155 mol) of 10% w/w aqueous sodium hypochlorite solution (previously adjusted to pH 9.5 using saturated sodium hydrogen carbonate) was added under nitrogen at a rate that maintained the reaction temperature below 15° C. Once addition was complete, the reaction mixture was stirred at 10-15° C. for 30 minutes. After this period, a further 5.7 Kg (7.7 mol) of 10% w/w aqueous sodium hypochlorite solution at pH 9.5 was added and the mixture was stirred for an additional 30 minutes at 10-15° C. The mixture was allowed to stand and the upper organic phase and the lower aqueous phase were separated. The aqueous phase was extracted by stirring for 30 minutes at 10-15° C. with 55 Kg of toluene. This extraction was repeated with a further 55 Kg of toluene and the organic phases thus obtained were combined with the organic phase obtained previously. Acidified potassium iodide solution (157 Kg; 2.4% w/w) was then added to the stirred, combined organic phases at 10-15° C. The organic phase turned deep orange during the addition. The mixture was stirred for a total of 30 minutes at 10-15° C. and subsequently the reddish, organic phase was separated. Sodium thiosulphate solution (167 Kg; 10% w/w aqueous) was next added to the stirred organic phase at 10-15° C. The organic phase turned from deep orange to colorless during the addition. The mixture was stirred for a total of 30 minutes at 10-15° C. and subsequently the pale yellow, organic phase was separated. Sodium hydrogen carbonate solution (157.9 Kg, 5% w/w) was next added to the stirred organic phase at 10-15° C. The resulting mixture was stirred for a total of 30 minutes at 10-15° C. and subsequently the pale yellow, organic phase was separated. Deionized water (158 Kg) was next added to the stirred organic phase at 10-15° C. The resulting mixture was stirred for a total of 30 minutes at 10-15° C. and subsequently the pale yellow, organic phase was separated. The organic phase was then concentrated by distilling off the toluene under vacuum at a temperature of ca. 40° C. This gave crude 3-[3-(trifluoromethyl)phenyl]propanal (Compound III) as a clear, yellow oil. The crude product was subsequently distilled under vacuum, collecting pure Compound III as a pale yellow oil in the temperature range of 85-105° C. at about 5 mbar. Yield: 26.0 Kg (83.1%).

### Example 4

### Preparation of Compound III (i.e., 3-(3-trifluoromethylphenyl) propanal

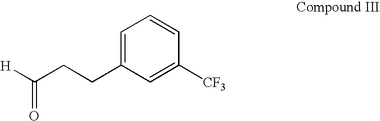

This example was carried out following the conditions of Example 3, but adding instead a total of 1.05 moles of sodium hypochlorite per mol of Compound (VII) in one single portion, Compound III was obtained with yield of 73.0%.

### Example 5

### Preparation of Compound III (i.e., 3-(3-trifluoromethylphenyl) propanal

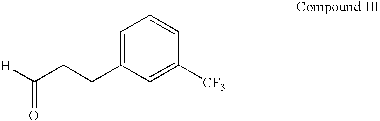

This example was carried out following the conditions of Example 3, but adding instead a total of 1.10 moles of sodium hypochlorite per mol of Compound (VII) in one single portion, Compound III was obtained with yield of 69.0%.

### Example 6

### Preparation of Compound III (i.e., 3-(3-trifluoromethylphenyl) propanal

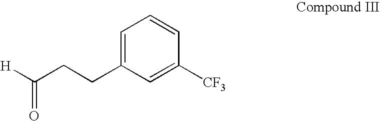

This example was carried out following the conditions of Example 3, but adding instead a total of 1.30 moles of sodium hypochlorite per mol of Compound (VII) in one single portion, Compound III was obtained with yield of 60.0%.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

What is claimed is:

1. A process for preparing 3-(3-trifluoromethylphenyl)propanal (Compound III) 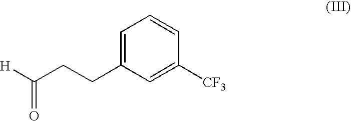
comprising oxidation of Compound VII 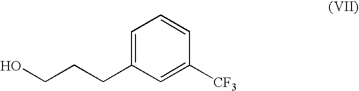
with an oxidizing agent using a nitroxyl compound as catalyst, in the presence of an inert solvent.

2. The process of claim 1, wherein said oxidizing agent is sodium hypochlorite.

3. The process of claim 2, wherein said sodium hypochlorite comprises approximately 1.05 moles of sodium hypochlorite per mole of Compound VII.

4. The process of claim 2, wherein said sodium hypochlorite is added in at least two portions.

5. The process of claim 4, wherein said sodium hypochlorite is added in two portions.

6. The process of claim 5, wherein a first portion of sodium hypochlorite comprises approximately 1.0 moles sodium hypochlorite per mole of Compound VII, and a second portion of sodium hypochlorite comprise approximately 0.05 moles of sodium hypochlorite per mole of Compound VII.

7. The process of claim 1, wherein said nitroxyl compound is TEMPO (2,2,6,6,-tetramethy-1-piperidinyloxi free radical).

8. The process of claim 1, further comprising the use of potassium bromide as a regenerating agent of the nitroxyl compound.

9. The process of claim 1, wherein said oxidation occurs at a temperature of approximately 5° C. to approximately 25° C.

10. The process of claim 1, wherein said oxidation occurs at a temperature of approximately 10° C. to approximately 15° C.

11. The process of claim 1, wherein said oxidation occurs at a temperature below approximately 15° C.

12. The process of claim 1, wherein said inert solvent is any solvent that does not take part in the reaction.

13. The process of claim 1, wherein said inert solvent is at least one of a cyclic alkane, an acyclic alkane, an aromatic solvent, a chlorinated solvent, an ester, an ether and mixtures thereof.

14. The process of claim 1, wherein said inert solvent is at least one of hexane, heptane, methylcyclohexane, toluene, dichloromethane, dichloroethane, chloroform, ethyl acetate, butyl acetate, isopropyl acetate, diethyl ether, tetrahydrofuran, tert-butyl methyl ether and mixtures thereof.

15. The process of claim 1, wherein said oxidation occurs for approximately 10 to approximately 60 minutes.

16. A process for preparing cinacalcet, its pharmaceutically acceptable salts and/or solvates thereof comprising converting Compound III made according to the processes of any of of claim 1 into cinacalcet, its pharmaceutically acceptable salts and/or solvates thereof.

17. Cinacalcet, its salts and/or solvates thereof prepared by the process of claim 16.

18. A formulation containing cinacalcet, its salts and/or solvates thereof according to claim 17.

19. A process for preparing Compound VII comprising:
i. reacting Compound IV, 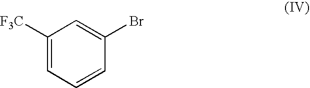
with Compound V, 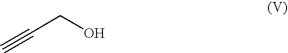
using 10% Pd/C catalyst, triphenyl phosphine, copper (I) iodide and diisopropylamine, to yield Compound VI; 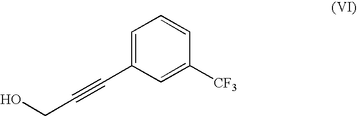
and
ii. converting Compound VI into Compound VII via catalytic hydrogenation.

20. The process of claim 1, wherein said Compound VII is prepared by a process comprising:
i. reacting Compound IV, 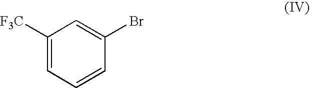
with Compound V, 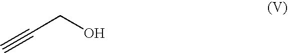
using 10% Pd/C catalyst, triphenyl phosphine, copper (I) iodide and diisopropylamine, to yield Compound VI; 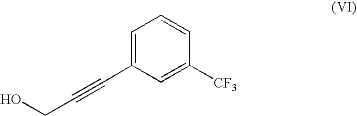
and
ii. converting Compound VI into Compound VII via catalytic hydrogenation.
